(19) United States
(12) Patent Application Publication
(10) Pub. No.: US 2013/0281702 A1
(43) Pub. Date: Oct. 24, 2013

(54) Methods For Preparing Fentanyl And Fentanyl Intermediates

(71) Applicants: Jonathan P. Pease, Cedarburg, WI (US); Anthony J. LePine, Greendale, WI (US); Catherine M. Smith, Grafton, WI (US)

(72) Inventors: Jonathan P. Pease, Cedarburg, WI (US); Anthony J. LePine, Greendale, WI (US); Catherine M. Smith, Grafton, WI (US)

(21) Appl. No.: 13/868,729

(22) Filed: Apr. 23, 2013

Related U.S. Application Data
(60) Provisional application No. 61/637,552, filed on Apr. 24, 2012.

(51) Int. Cl. C07D 211/58

(52) U.S. Cl. CPC C07D 211/58
USPC 546/244

(57) ABSTRACT

A method and an intermediate are provided for preparing fentanyl. Aniline and 1-phenylethyl-4-piperidone are reacted with a borane complex, in a lower C₁-C₄ alcoholic solvent, in the presence of an alkanoic acid. The reaction mixture is then treated with a hydrohalic acid to precipitate 4-anilino-N-phenethyl-4-piperidine (ANPP) as the bis-hydrohalide salt in high yield and purity. This ANPP salt may be directly treated with propionyl halide to produce fentanyl, or the ANPP salt may be converted to the free base of ANPP and similarly treated with propionyl halide to produce fentanyl.

### CROSS-REFERENCES TO RELATED APPLICATIONS

This application claims priority from U.S. Provisional Patent Application No. 61/637,552, filed Apr. 24, 2012.

### STATEMENT REGARDING FEDERALLY SPONSORED RESEARCH

Not Applicable.

### BACKGROUND OF THE INVENTION

1. Field of the Invention

The invention relates to methods and intermediates for preparing fentanyl and the fentanyl precursor ANPP (4-anilino-N-phenethyl-4-piperidine or 1-phenethyl-N-phenylpiperidin-4-amine) in high purity and high yield, particularly in commercial quantities.

2. Description of the Related Art

Fentanyl is a potent, synthetic opioid analgesic. Fentanyl is typically used to treat sudden episodes of pain, and is available by prescription in various forms including a lozenge on a handle, a sublingual tablet, a film, and a buccal tablet. It is also administered by intravenous injection in a hospital setting.

Although various methods for preparing fentanyl have been described in the art, there is a continuing need for improved methods for preparing fentanyl and fentanyl intermediates, particularly in commercial quantities with high yield and high purity.

### SUMMARY OF THE INVENTION

The foregoing needs are met by a method according to the invention for preparing fentanyl and the fentanyl precursor ANPP.

The method for preparing the fentanyl precursor ANPP includes the steps of (a) reacting aniline with a compound of formula (IV)

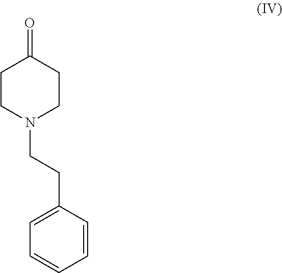 [SMILES: O=C1CCN(CCc2ccccc2)CC1]

and
(b) adding a complex of formula D→A to produce the compound of formula (II) or a salt thereof:

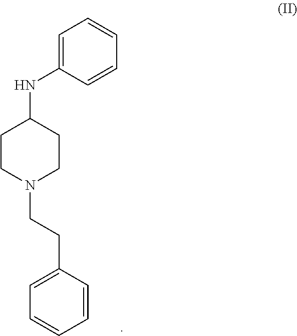 [SMILES: c1ccc(CCN2CCC(Nc3ccccc3)CC2)cc1]

In the complex, D can be a group of atoms having an electron donor atom, and A can have the formula (III):

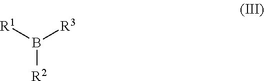

wherein R₁, R₂, and R₃ are independently selected from hydrogen, substituted or unsubstituted C₁-C₁₀ alkyl, substituted or unsubstituted C₁-C₁₀ alkoxy, aryloxy, and wherein at least one of R₁, R₂, and R₃ is hydrogen. Preferably, R₁, R₂, and R₃ are hydrogen. D can be heterocyclic, and the electron donor atom can be selected from nitrogen, oxygen, or sulfur. Preferably, the electron donor atom is nitrogen. Most preferably, D is selected from substituted or unsubstituted pyridine, substituted or unsubstituted aniline, or substituted or unsubstituted amine. In one version of the method, D is 5-ethyl-2-methylpyridine, and R₁, R₂, and R₃ are hydrogen.

In the method for preparing the fentanyl precursor ANPP, a C₁-C₄ alcoholic solvent and/or an organic acid can be added with the complex. The organic acid can be an alkanoic acid of C₁-C₈. Preferably, the organic acid is acetic acid (ethanoic acid).

In the method for preparing the fentanyl precursor ANPP, a mineral acid can be added at the completion of the reaction thereby crystallizing out a salt of the compound of formula (II). Preferably, the mineral acid is a hydrohalic acid. Most preferably, the mineral acid is hydrochloric acid.

The method for preparing fentanyl includes the steps of (a) reacting aniline with a compound of formula (IV) as shown above; and (b) adding a complex of formula D→A, wherein D is a group of atoms having an electron donor atom, and A has formula (III) as shown above wherein R₁, R₂, and R₃ are independently selected from hydrogen, substituted or unsubstituted C₁-C₁₀ alkyl, substituted or unsubstituted C₁-C₁₀ alkoxy, aryloxy, and wherein at least one of R₁, R₂, and R₃ is hydrogen; and (c) adding a propionyl halide or propionic anhydride to produce the compound of formula (I):

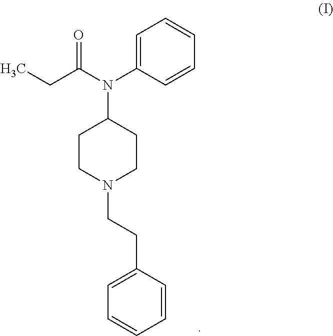 [SMILES: CCC(=O)N(c1ccccc1)C1CCN(CCc2ccccc2)CC1]

In the complex used in the method for preparing fentanyl, D can be a group of atoms having an electron donor atom, and A can have the formula (III):

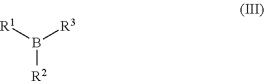

wherein R₁, R₂, and R₃ are independently selected from hydrogen, substituted or unsubstituted C₁-C₁₀ alkyl, substituted or unsubstituted C₁-C₁₀ alkoxy, aryloxy, and wherein at least one of R₁, R₂, and R₃ is hydrogen. Preferably, R₁, R₂, and R₃ are hydrogen. D can be heterocyclic, and the electron donor atom can be selected from nitrogen, oxygen, or sulfur. Preferably, the electron donor atom is nitrogen. Most preferably, D is selected from substituted or unsubstituted pyridine, substituted or unsubstituted aniline, or substituted or unsubstituted amine. In one version of the method, D is 5-ethyl-2-methylpyridine, and R₁, R₂, and R₃ are hydrogen.

In the method for preparing fentanyl, a mineral acid can be added after adding a complex of formula D→A thereby crystallizing out a salt of the compound of formula (II) as shown above. Preferably, the mineral acid is a hydrohalic acid. Most preferably, the mineral acid is hydrochloric acid.

In the method for preparing fentanyl, a propionyl halide is preferably used in step (c) to produce the compound of formula (I). Most preferably, the propionyl halide is propionyl chloride.

The methods of the invention have many advantages, including without limitation: (i) the reduction of the imine of the intermediate with a wide range of boranes (e.g., 5-ethyl-2-methylpyridine borane, picoline borane, pyridine borane, triethylamine borane, diethylaniline borane); (ii) the imine of the intermediate is reduced in situ, with no need for separate generation or isolation of the imine or a workup procedure; (iii) the intermediate of Formula (II) can be isolated directly from the reaction mixture as a salt (e.g., the bis-HCl salt) with no need for an aqueous workup; (iv) there is no need for a cycle of free-basing/salt formation; (v) the method is a scalable, high yielding and high purity process with easy to handle commercially available reagents; (vi) the reduction of the imine is performed in an environmentally friendly lower alcohol; and (vii) the salt of Formula (II) can be converted to fentanyl with no need for a separate generation or isolation of the freebase.

These and other features, aspects, and advantages of the present invention will become better understood upon consideration of the following detailed description, drawings, and appended claims.

### BRIEF DESCRIPTION OF THE DRAWINGS

FIG. 1 shows one example version of a reaction scheme according to the invention for the preparation of fentanyl and a fentanyl intermediate.

### DETAILED DESCRIPTION OF THE INVENTION

This invention provides a method for preparing fentanyl which has the formula (I).

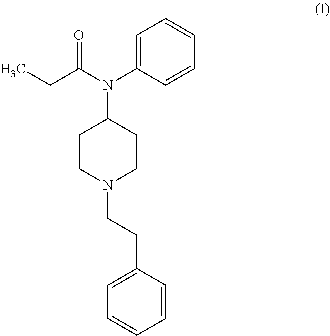 [SMILES: CCC(=O)N(c1ccccc1)C1CCN(CCc2ccccc2)CC1]

Fentanyl is also known as N-(1-(2-phenylethyl)-4-piperidinyl)-N-phenylpropanamide.

This invention also provides a method for preparing a compound of formula (II).

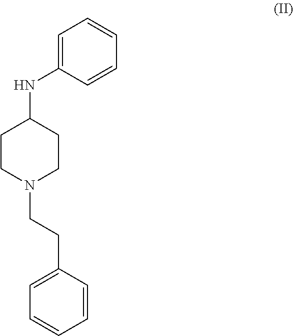 [SMILES: c1ccc(CCN2CCC(Nc3ccccc3)CC2)cc1]

Formula (II) is also known as 4-Anilino-N-phenethyl-4-piperidine or ANPP. The compound of formula (II) is an intermediate that can be used to produce fentanyl.

### Example Reaction Conditions for Synthesizing the Compounds of Formulas (I) & (II)

Scheme 1 below is an example scheme for Formula II (ANPP) synthesis:

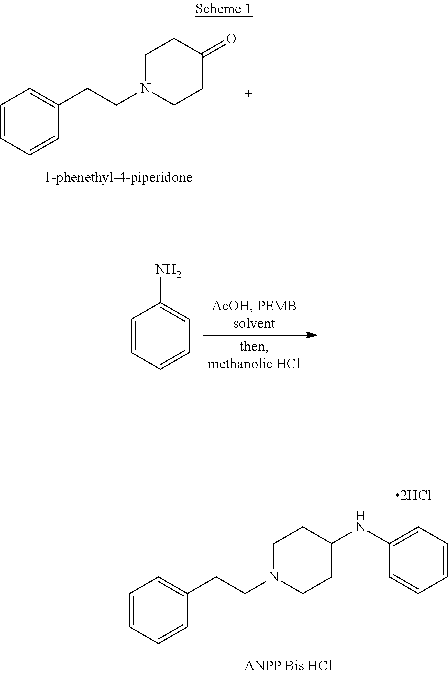 [SMILES: O=C1CCN(CCc2ccccc2)CC1 ; Nc1ccccc1 ; c1ccc(CCN2CCC(Nc3ccccc3)CC2)cc1]

### Example Reductive Amination Ranges for ANPP:

Several borane complexes have been shown to be effective reducing agents. These include 5-ethyl-2-methylpyridine borane, pyridine borane, picoline borane, triethylamine borane, and diethylaniline borane. 5-ethyl-2-methylpyridine borane (PEMB) is the preferred reagent based on ease of handling, stability, and commercial availability.

Solvents include alcohols from 1-4 carbons, such as methanol, ethanol, propanol, isopropanol, and butanol, with either methanol or isopropanol being the preferred solvents.

The amount of borane complex equivalents can range from 0.5 to 1.1 molar equivalents and shows some dependence on the solvent.

The amount of acetic acid used in the reaction can vary from between 0.5 to 3.0 molar equivalents, with 2 molar equivalents being the preferred amount.

The reaction has been shown to be amenable to being run at temperatures between 0° C. to 55° C.

Anhydrous solutions of hydrochloric acid (HCl) in various alcoholic solvents such as methanol, ethanol, and isopropanol were found to be effective at quenching the reaction and generating the desired salt. Other solutions of HCl in organic solvents such as diethyl ether and dioxane are expected to be effective. The amount of HCl used to quench residual borane and generate the Bis HCl salt can range between 3.0 to 4.0 equivalents.

Range of amine substituent can be 0.95 to 1.2 equivalents, preferably 1.05.

The yields for the Formula II (ANPP) synthesis of can be 60% or more, 70% or more, 80% or more, 85% or more, or 90% or more.

Example Fentanyl Chemistry Routes with ANPP Bis HCl Salt:

The isolated ANPP Bis HCl salt can be taken directly on to form fentanyl in a single pot reaction without isolation of the ANPP freebase. A few potential routes are shown below.

Scheme 2 below is an example scheme for the conversion of ANPP Bis HCl to Formula (I) (fentanyl).

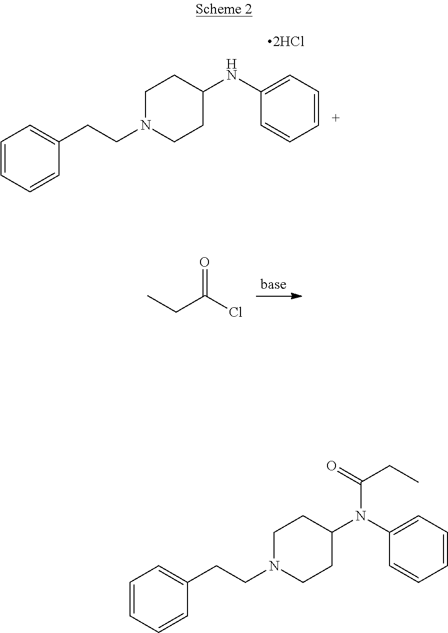

One route to synthesize fentanyl is to react ANPP Bis HCl with propionyl chloride in the presence of an organic base such as triethylamine or pyridine in an appropriate solvent such as CH₂Cl₂, ethyl acetate (EtOAc), methyl tert-butyl ether (MTBE), tetrahydrofuran (THF), or toluene, etc.

Another route utilizes an inorganic base such as KOH, NaOH, or K₂CO₃ with propionyl chloride in a mixture of water and an appropriate organic solvent such as CH₂Cl₂, toluene, or THF, etc.

The yields for the Formula I (fentanyl) synthesis can be 60% or more, 70% or more, 80% or more, 85% or more, or 90% or more. The purity can be >99% pa by HPLC.

Example Fentanyl Chemistry Routes with ANPP Freebase

Scheme 3 is an example scheme for the conversion of ANPP freebase to Formula I (fentanyl).

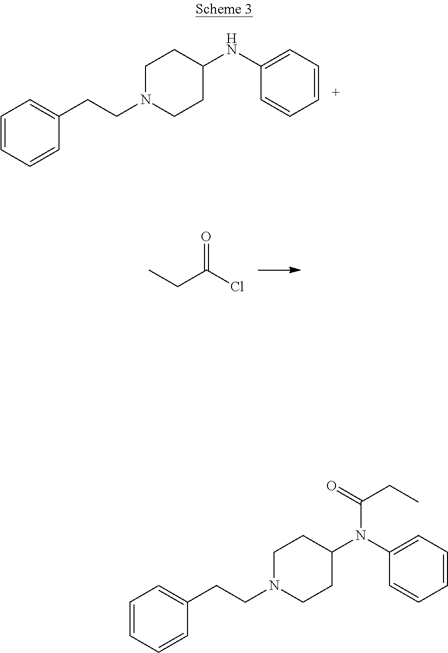

ANPP freebase can be reacted with propionyl chloride with or without an organic base such as triethylamine or pyridine in an appropriate solvent such as CH₂Cl₂, EtOAc, MTBE, THF, or toluene, etc.

Another route utilizes an inorganic base such as KOH, NaOH, or K₂CO₃ with propionyl chloride in a mixture of water and appropriate organic solvent such as CH₂Cl₂, or THF, etc.

Propionic anhydride can be used as the acylating agent with or without 4-dimethylaminopyridine (DMAP) in an appropriate solvent such as CH₂Cl₂, EtOAc, MTBE, THF, or toluene, etc.

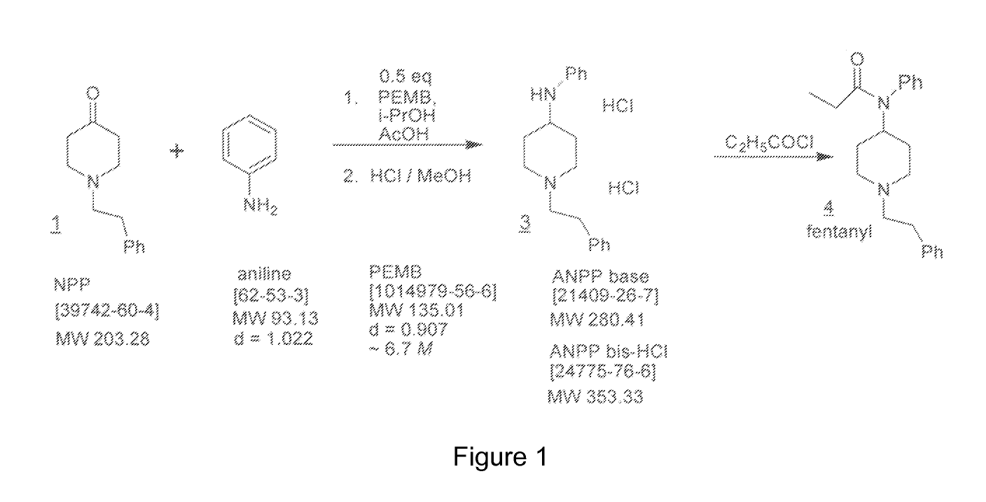

One non-limiting example version of a method of the invention is shown in FIG. 1 in which CAS numbers are in brackets, MW is molecular weight, d is density, and M is molarity. 1-phenethyl-4-piperidone (NPP) is reacted with aniline in the presence of 5-ethyl-2-methylpyridine borane, isopropanol, and acetic acid. A solution of hydrochloric acid in methanol quenches the reaction and generates 4-Anilino-N-phenethyl-4-piperidine (ANPP) bis-HCl salt. The ANPP bis-HCl salt is reacted with propionyl chloride for produce fentanyl.

### EXAMPLES

The following Examples have been presented in order to further illustrate the invention and are not intended to limit the invention in any way.

### Example 1

### 4-Anilino-N-phenethyl-4-piperidine Bis-HCl (ANPP Bis-HCl salt) using PEMB

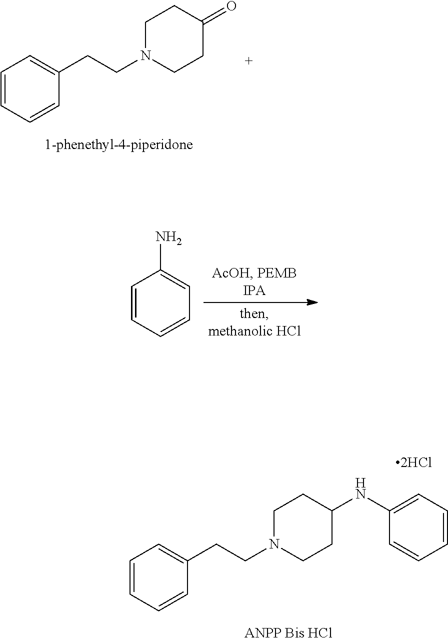 [SMILES: O=C1CCN(CCc2ccccc2)CC1 ; Nc1ccccc1 ; c1ccc(CCN2CCC(Nc3ccccc3)CC2)cc1]

To a solution of 1-phenethyl-4-piperidone (10.0 g, 49.2 mmol) in isopropanol (IPA) (80 mL) was added aniline (4.7 mL, 52 mmol), and the solution stirred for 5 minutes. Acetic acid (5.6 mL, 98 mmol) was added, followed by feeding in 5-ethyl-2-methylpyridine borane complex (PEMB) (4.0 mL, 27 mmol). The solution was stirred for 4 hours before quenching by feeding in a 4M solution of HCl in methanol (44 mL). The resulting slurry was stirred 2 hours before filtering, washing the solid with IPA, a 3:1 Solution of IPA:MTBE and MTBE. The solid was dried to recover 14.7 g of the product as a white solid (85% yield).

### Example 2

### Synthesis of ANPP Bis-HCl Using PEMB in MeOH

To a solution of 1-phenethyl-4-piperidone (5.0 g, 24.6 mmol) in methanol (MeOH) (60 mL) was added aniline (2.35 mL, 25.7 mmol), and the solution stirred for 5 minutes. Acetic acid (2.8 mL, 49 mmol) was added, followed by feeding in 5-ethyl-2-methylpyridine borane complex (3.1 mL, 21 mmol). The solution was stirred for 22 hours before quenching by feeding in a 4M solution of HCl in methanol (22 mL). The resulting slurry was stirred a minimum of 2 hours before filtering, washing the solid with IPA, a 3:1 solution of IPA:MTBE, and MTBE. The solid was dried to recover 7.88 g of the product as a white solid (91% yield).

### Example 3

### Synthesis of ANPP Bis-HCl Using Pyridine Borane Complex

Following the procedure given in Example 1 using pyridine borane complex gave ANPP Bis-HCl as a white solid in 90.8% yield.

### Example 4

### Synthesis of ANPP Bis-HCl Using 2-picoline Borane Complex

Following the procedure given in Example 1 using 2-picoline borane complex gave ANPP Bis-HCl as a white solid in 90.2% yield.

### Example 5

### Synthesis of ANPP Bis-HCl Using Triethylamine Borane

Following the procedure given in Example 1 using triethylamine borane complex gave ANPP BisHCl as a white solid in 69.5% yield.

### Example 6

### Synthesis of ANPP Bis-HCl Using Diethylaniline Borane Complex

Following the procedure given in Example 1 using diethylaniline borane complex gave ANPP Bis HCl as a white solid in 79.9% yield.

### Example 7

### N-(1-(2-phenylethyl)-4-piperidinyl)-N-phenylpropanamide (Fentanyl)

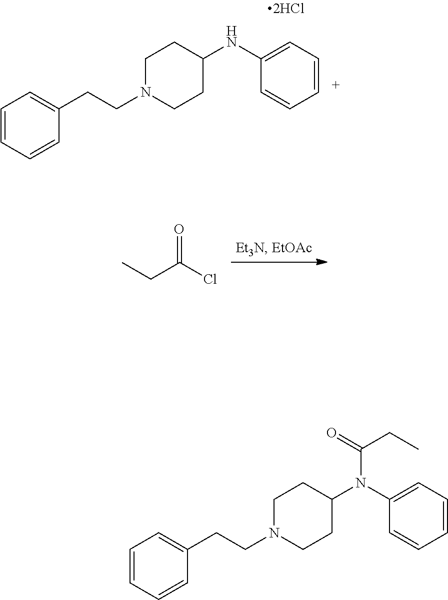

To a slurry of ANPP Bis HCl (12.0 g, 33.9 mmol) in EtOAc (108 mL) was charged triethylamine (11.8 mL, 84.7 mmol). The slurry was heated to 55° C. and propionyl chloride (5.2 mL, 59.8 mmol) was fed in over 3 minutes. The reaction was heated to reflux and held for 2 hours. The slurry was cooled to room temperature followed by the addition of 70 ml of water and the pH adjusted to 8-9 using a solution of NaOH (10% by wt in water). The layers were separated, and the organic layer was washed with a saturated NaHCO₃ solution, water and brine, and then dried using Na₂SO₄. The solution was filtered and distilled under vacuum to remove most of the solvent. MTBE was charged and the solution distilled under vacuum to a final volume of 17 mL. Heptane (45 mL) was added and the resulting slurry cooled to ° C. The solids were filtered and washed with heptane. The product was dried to recover 8.24 g of fentanyl as a white solid which is a yield of 72° A).

### Example 8

### Fentanyl

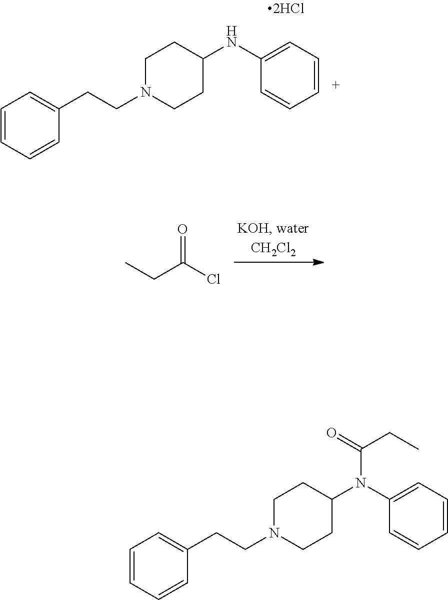

To a slurry of ANPP Bis HCl (6.0 g, 17.0 mmol) in CH₂Cl₂ (78 mL) was charged a solution of 2.8M KOH in water. Once the solids dissolved, propionyl chloride (3.0 mL, 34.5 mmol) was added and the reaction stirred for 2 hours. The pH of the aqueous layer was adjusted to ≧8 using 5% NaOH solution, and the layers separated. The organic layer was washed with water and brine, and then dried using Na₂SO₄. The solution was filtered and the solvent removed under vacuum. The crude fentanyl was dissolved in MTBE, and the solution was distilled to a volume of 10 mL to which was added 30 mL of heptane. The resulting slurry was cooled to ≦5° C., filtered and the product washed with heptane. The product is dried to recovered 3.85 g of fentanyl as a white solid, 67.2° A) yield.

Thus, the invention provides improved methods and intermediates for preparing fentanyl in high purity and high yield, particularly in commercial quantities.

Although the present invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A method for preparing a compound of formula (II) or a salt thereof: 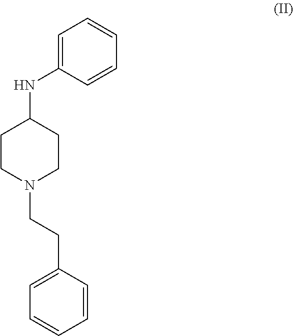 [SMILES: c1ccc(CCN2CCC(Nc3ccccc3)CC2)cc1]
the method comprising:
(a) reacting aniline with a compound of formula (IV) 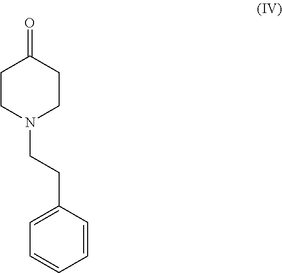 [SMILES: O=C1CCN(CCc2ccccc2)CC1]
and
(b) adding a complex of formula D→A to produce the compound of formula (II), wherein D is a group of atoms having an electron donor atom, and A has formula (III): 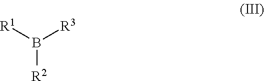
wherein R₁, R₂, and R₃ are independently selected from hydrogen, substituted or unsubstituted C₁-C₁₀ alkyl, substituted or unsubstituted C₁-C₁₀ alkoxy, aryloxy, and wherein at least one of R₁, R₂, and R₃ is hydrogen.

2. The method of claim 1 wherein:
D is heterocyclic.

3. The method of claim 2 wherein:
the electron donor atom is selected from nitrogen, oxygen, or sulfur.

4. The method of claim 2 wherein:
the electron donor atom is nitrogen.

5. The method of claim 1 wherein R₁, R₂, and R₃ are hydrogen.

6. The method of claim 1 wherein:
D is selected from substituted or unsubstituted pyridine, substituted or unsubstituted aniline, or substituted or unsubstituted amine.

7. The method of claim 1 wherein:
D is 5-ethyl-2-methylpyridine, and
R₁, R₂, and R₃ are hydrogen.

8. The method of claim 1 further comprising:
adding a C₁-C₄ alcoholic solvent in step (b).

9. The method of claim 1 further comprising:
adding an organic acid in step (b).

10. The method of claim 8 wherein:
the organic acid is an alkanoic acid of C₁-C₈.

11. The method of claim 8 wherein:
the organic acid is acetic acid.

12. The method of claim 1 further comprising:
(c) adding a mineral acid at completion of reaction thereby crystallizing out a salt of the compound of formula (II).

13. The method of claim 12 wherein:
the mineral acid is a hydrohalic acid.

14. The method of claim 12 wherein:
the mineral acid is hydrochloric acid.

15. A method for preparing a compound of formula (I): 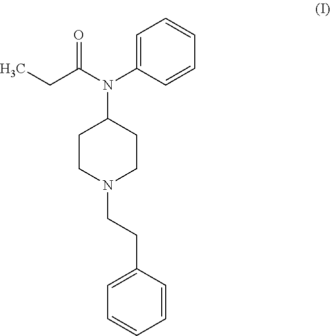 [SMILES: CCC(=O)N(c1ccccc1)C1CCN(CCc2ccccc2)CC1]
the method comprising:
(a) reacting aniline with a compound of formula (IV) 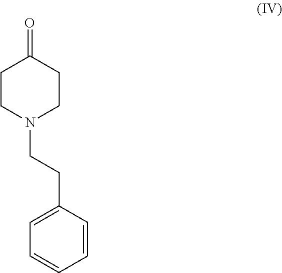 [SMILES: O=C1CCN(CCc2ccccc2)CC1]
and
(b) adding a complex of formula D→A, wherein D is a group of atoms having an electron donor atom, and A has formula (III): 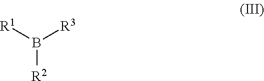
wherein R₁, R₂, and R₃ are independently selected from hydrogen, substituted or unsubstituted C₁-C₁₀ alkyl, substituted or unsubstituted C₁-C₁₀ alkoxy, aryloxy, and wherein at least one of R₁, R₂, and R₃ is hydrogen; and
(c) adding a propionyl halide or propionic anhydride to produce the compound of formula (I).

16. The method of claim 15 wherein:
step (b) further comprises adding a mineral acid thereby crystallizing out a salt of a compound of formula (II):

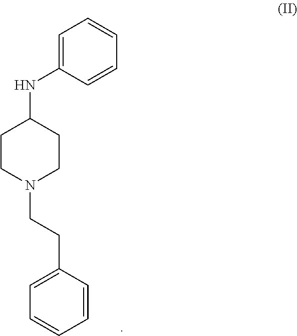 [SMILES: c1ccc(CCN2CCC(Nc3ccccc3)CC2)cc1]

17. The method of claim 16 wherein the mineral acid is a hydrohalic acid.

18. The method of claim 15 wherein:
the propionyl halide is propionyl chloride.

19. The method of claim 15 wherein:
D is heterocyclic, and the electron donor atom is nitrogen.

20. The method of claim 15 wherein:
D is selected from substituted or unsubstituted pyridine, substituted or unsubstituted aniline, substituted or unsubstituted amine.

21. The method of claim 15 wherein:
D is 5-ethyl-2-methylpyridine, and
R₁, R₂, and R₃ are hydrogen.
